(19) United States
(12) Patent Application Publication
(10) Pub. No.: US 2008/0131388 A1
(43) Pub. Date: Jun. 5, 2008

(54) COSMETIC PRODUCT

(75) Inventor: Masakazu KOMURO, Tokyo (JP)

Correspondence Address: OBLON, SPIVAK, MCCLELLAND MAIER & NEUSTADT, P.C., 1940 DUKE STREET, ALEXANDRIA, VA, 22314, US

(73) Assignee: NAC CO., LTD., Setagaya-ku (JP)

(21) Appl. No.: 11/924,188

(22) Filed: Oct. 25, 2007

(30) Foreign Application Priority Data
Nov. 30, 2006 (JP) 2006-323438

(51) Int. Cl. A61K 8/19; A61Q 1/00

(52) U.S. Cl. 424/69

(57) ABSTRACT

A cosmetic product is provided, which is directed at improving infrared radiation effects, charged particle emission and ultraviolet blocking effects. Diamond powder formed by shockwaves is suitably blended with a cosmetic product and specific infrared wavelengths and charged particle emission characteristics generated by the semiconductor characteristics thereof are used. An inorganic oxide powder may be added as needed, so as to produce characteristics that are beneficial for cosmetic materials such as foundations, powders, gels, lipsticks, eye shadows, and manicure products.

### TECHNICAL FIELD

The present invention relates to a cosmetic product using diamond powder, and in particular to a product that is intended to be applied to the body.

### BACKGROUND ART

Conventionally, the value of diamond as a gemstone has been exploited for cosmetic materials by mixing it with enamels or the like and applying it to the nails or the like, in order to form decorative coatings. Consequently, the sizes of the diamonds that have been used have been from 0.0033 to 0.01 carats (see JP-2005-102980-A).

Because the diamonds have conventionally found application as decoration, they have been cut into polyhedrons, with the objective of amplifying their brightness as gemstones, so as to reflect and refract light in various directions from each of the surfaces when the gemstone is struck by light.

It also been proposed that garnets, rubies, sapphires, emeralds and tourmalines that have been cut into polyhedrons be used as cosmetic product materials, in place of diamonds. It should be noted that all of these are used as decorative products.

Furthermore, cosmetic products are known wherein titanium oxide, zinc oxide and the like, which are ceramic powders, are blended with cosmetic bases such as oils, waxes and higher alcohols. It should be noted that these ceramic powders are used with the objective of preventing sunburn by blocking ultraviolet radiation, by making use of ultraviolet reflection capacities based on their band structures as insulators (see JP-2000-119152-A and JP-2002-154915-A).

Products using tourmaline and titanium oxide are known as cosmetic products that make use of an infrared emission effect but, as both of these are insulators, the infrared emission effect thereof is weak. Furthermore, while tourmaline is a piezoelectric-pyroelectric material, it is an insulator and therefore, even if it is in contact with the body as a cosmetic product, a charge transfer effect cannot be expected, and thus there is substantially no possibility of generating a negative charge effect (see JP-2003-192527-A).

The present invention is a reflection of the prior art described above and a problem to be solved by the invention is that of providing a cosmetic product which, when used as a cosmetic product, simultaneously exerts the triple effects of blocking ultraviolet radiation, emitting infrared radiation and emitting charged particles, which have the beautifying effect of preventing sunburn and which, by way of activation resulting from promotion of the skin metabolism as a result of increased blood circulation due to penetration by infrared radiation and charged particles, not only prevent deterioration of the skin, but exert beautifying effects.

### DISCLOSURE OF THE INVENTION

In order to achieve the object described above, the present invention is a cosmetic product comprising diamond powder having a nano-sized SP3 structure formed by shockwaves (claim 1).

Preferably, an infrared emitting material other than the diamond powder formed by shockwaves and powdered semiconductor material are further blended with the cosmetic product (claim 2).

One or more of tourmaline, quartz and graphite can be used as the infrared emitting material other than the diamond powder formed by shockwaves and germanium and/or silicon can be used as the powdered semiconductor material (claim 3).

Preferably, a powdered composite material containing no less than 0.1 wt % and no more than 10 wt % of diamond powder is used for the cosmetic product of the present invention (claim 4).

Because nitrogen and the like from explosives used in the manufacturing process for the diamond powder formed by shockwaves enters the crystal lattice of the diamond, a semiconductor results having a 5.5 eV bandwidth with a 0.1 to 0.4 eV impurity level. Consequently, when the cosmetic product is in contact with the human body, it emits infrared radiation at wavelengths of 4 to 10 μm, which is effective in increasing blood flow in the human body as a result of thermal excitation at the level of body heat, and charged particles are emitted from the excited impurity level. The negatively charged particles released penetrate the human body by way of the skin so as to promote blood flow and activate the skin from the interior of the body. Accordingly, the compatibility of the cosmetic product with the human body is improved, and absorption of the active ingredients in the cosmetic product is promoted. As described above, the cosmetic product of the present invention simultaneously exerts the triple effects of ultraviolet blocking, infrared emission and charged particle emission, allowing it to exert a beautifying effect by preventing sunburn and, by way of activation resulting from promotion of the skin metabolism as a result of increased blood circulation due to penetration by infrared radiation and charged particles, an effect that not only prevents deterioration of the skin, but exerts a beautifying effect.

By virtue of the invention recited in claims 2 and 3, the effective infrared radiation bandwidth is expanded, and the effect thereof is more fully exerted.

By virtue of the invention in claim 4, a cosmetic product is produced that is capable of emitting a sufficient amount of charged particles as a result of thermal excitation at the body temperature level.

### BRIEF DESCRIPTION OF THE DRAWINGS

FIG. 1 is an infrared emission spectrogram for the diamond particles used in the present invention and market available tourmaline.

FIG. 2 is a graph of thermal characteristics for diamond charged particle emission.

### BEST MODE FOR CARRYING OUT THE INVENTION

Next, modes of embodiment of the present invention will be described.

Due to their band structure, diamonds formed by shockwaves absorb thermal energy resulting from body heat, optical energy in a broad range of wavelengths from sunlight, ultraviolet radiation and the like, and convert this into infrared radiation emitted from the impurity donor level at wavelengths of 4 to 10 μm, which is beneficial for the human body, and also have an ultraviolet blocking effect. Consequently, in order to expand the effective infrared radiation bandwidth and consequently further exert the effect thereof, it is possible to make joint use of semiconductors such as germanium having narrow bandwidths and emitting long wavelengths of approximately 100 μm, or piezoelectric/pyroelectric materials such as tourmaline, which output infrared at the relatively short wavelengths of no greater than 4 μm.

Because diamond is more expensive than insulating ceramics such as tourmaline, the ratio at which this is used may be suitably selected according to the infrared wavelength band and the charged particle emission capacity required. In order to produce a cosmetic product that is capable of emitting sufficient amounts of charged particles by way of thermal excitation at the body temperature level, the minimum amount of diamond added is preferably no less than 1 wt % and no greater than 10 wt % of the piezoelectric/pyroelectric materials and powdered semiconductor. At less than 1 wt %, the infrared and charged particle effects specific to diamond are reduced. At greater than 10 wt %, there is a tendency for degradation of the oils, higher alcohols and the like which serve as the cosmetic base, as a result of the charged particles emitted from the diamond.

The amount of diamond, piezoelectric/pyroelectric materials and powdered semiconductor (powdered composite material) added is preferably no less than 5 wt % and no greater than 20 wt % of the cosmetic product composition. At less than 5 wt %, the charged particle emission effect resulting from the diamond is lowered, and the effect of activating the skin cannot be expected. Conversely, at greater than 20 wt %, due to the charged particles from diamond, the bonds in the organic macromolecular materials constituting the cosmetic product are broken, which tends to reduce shelf life and increase costs.

According to the present invention, a cosmetic product is caused to contain diamond powder formed by shockwaves, and when this cosmetic product is used on the human body, as a result of the infrared radiation from the material and energy possessed by the anionic charged particles, cells in the human body are activated, which is to say the infrared radiation, centered around 4 to 10 μm, causes heat to penetrate deep into the interior of the human body, and by acting on water and cells, interactively activates the molecules, promoting a micro massage effect and a self-heating effect.

Furthermore, the charged particles penetrate to the interior of the human body by way of the skin, having an effect of helping muscles to recover from fatigue and reducing pain and, therefore, when used in the cosmetic product, they activate the skin, so that the effect of the cosmetic product is enhanced, and the active ingredients in the cosmetic product readily permeate the skin, which not only has a therapeutic effect, but also has major beautifying effects such as improving skin elasticity.

The diamond powder formed by shockwaves is fine, having a particle sizes of 0.1 to 0.01 μm (10 to 100 nm) and is readily dispersed in cosmetic bases, and therefore this can be included in basic cosmetics such as powders, creams, gels, face masks and foundations, as well as makeup cosmetic products, in combination with other piezoelectric/pyroelectric materials such as tourmaline, as necessary, by mixing this with an emulsifying agents and the like. When this cosmetic product is applied to the skin, it can be expected to have a significant effect on the human body.

Furthermore, because the surface of the diamond powder is coated with a thin SP² graphite layer, it disperses readily into cosmetic bases, and because the powder is of a fine nano-size, it can penetrate the skin by way of hair follicles and the like in the skin, and activate the skin by way of the infrared radiation wavelengths and radiated anions. Furthermore, the anions have a reducing effect, which activates skin that has been damaged by oxidization resulting from ultraviolet radiation and the like, and repairs it.

The basic particle size of the diamond powder formed by shockwaves is 10 to 100 nm, and these are normally present as aggregates of approximately 1 to 4 μm. If the infrared emitting material is an inorganic particle such as tourmaline, after blending the two at a predetermined ratio, in order to disperse them more uniformly, a suspension is produced in a solvent such as water containing a dispersant, this is mixed by way of agitation, and the suspension is sprayed out of a pressure spray nozzle by way of hot air, so as to produce a unified composite powder, which is mixed with a cosmetic base in order to produce the cosmetic product.

These composite particles can be used in general cosmetic products, and can be blended with cosmetic materials such as foundations, face powders, foundation creams, eye shadows, eyeliners, lipsticks, manicure products, emulsions, creams and the like. There are no limits on the amount of composite powder that can be blended with the cosmetic material, but this is preferably 5 to 20 wt %. At less than 5 wt %, the charged particle emission effect resulting from the diamond is lowered, and the effect of activating the skin cannot be expected. Conversely, at greater than 20 wt %, due to the charged particles from diamond, the bonds in the organic macromolecular materials constituting the cosmetic product are broken, which tends to reduce shelf life and increase costs. In the case of powdered cosmetic products that do not use polymer materials, the upper limit for the amount blended is determined by cost and the compatibility of the cosmetic product with the skin.

### Embodiments

Specific examples of methods for producing the diamond particles used in the present invention include exploding a high-performance CB explosive in a sealed container so as to momentarily generate pressures of 2,000,000 atmospheres and temperatures of several thousand degrees, so as to form carbon particles having an SP³ structure, or placing fine carbon powder, copper powder and the like in a sealed container, and igniting explosives that have been placed on top of these, so as to subject the powder mixture to similar pressures and temperatures, and after converting the carbon to a crystalline structure, dissolving the metal powder with acid so as to produce carbon particles having an SP³ structure. In the cooling process after producing these particles, the surface may be covered with a carbon film having an SP² structure, but if necessary this SP² structure carbon film can be removed with heated concentrated nitric acid or heated supercritical water. (See Eiji Osawa, Japan Nanonet Bulletin, pp. 108 Jun. 3, 2008 and Sumitomo Coal Mining Co., Ltd, Cluster Technology Research Group, Jun. 3, 2027)

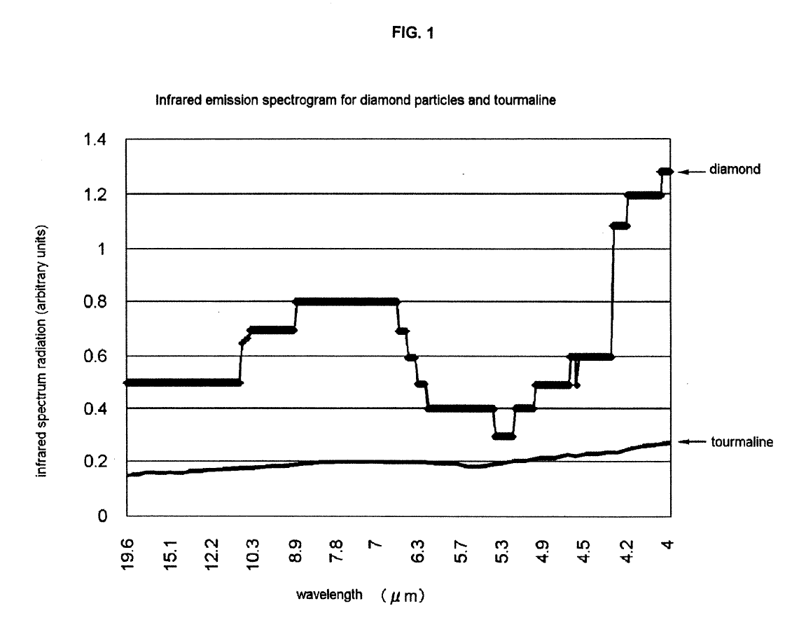

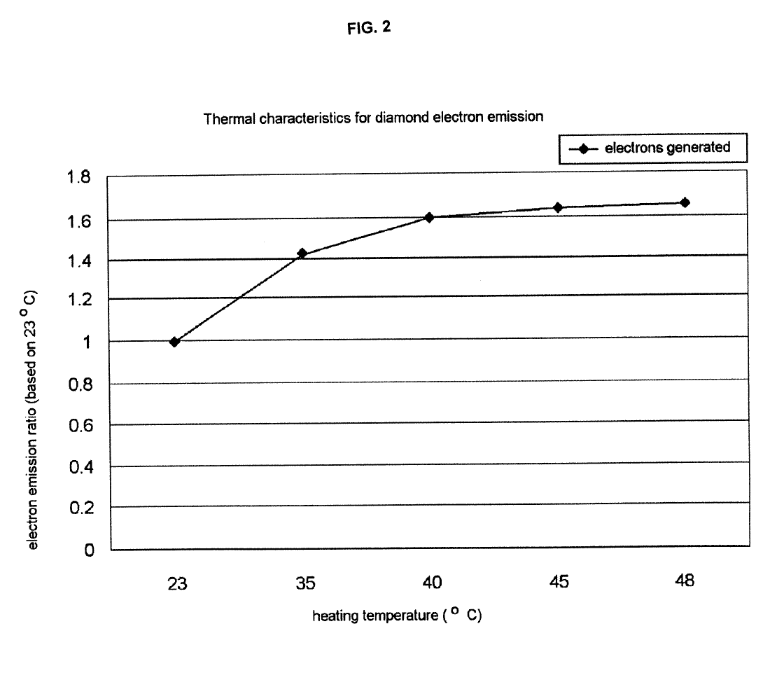

The infrared wavelength emission characteristics of diamond particles produced by the method described above and that of market available tourmaline particles, as well as the charged particle emission temperature characteristics of the diamond particles are shown in FIGS. 1 and 2. FIG. 1 is an infrared emission spectrogram for the diamond powder used in the present invention and market available tourmaline. In the working example, the diamond powder was blended at 10 wt % with respect to the emulsifier, and in the comparative example the tourmaline powder was blended at 50 wt % with respect to the emulsifier. Because tourmaline is an electrical insulator, it emits substantially no charged particles as a result of thermal excitation at body temperature levels. As compared to this, diamond formed by shockwaves is a semiconductor, and therefore large amounts of excited particles are emitted from the donor level.

As shown in FIG. 1 and FIG. 2, the carbon particles comprising SP³ diamond formed by shockwaves are activated by solar energy and body heat, and as a result of the specific infrared wavelengths emission and charged particle penetration effect that result from the excited charged particles, human skin is activated so as to improve the compatibility of the cosmetics while exerting a great whitening effect as a result of the light reflecting characteristics of the diamonds.

1. A cosmetic product comprising diamond powder formed by shockwaves which is capable of emitting an infrared and a charged particle.

2. The cosmetic product recited in claim 1, wherein an infrared emitting material other than the diamond powder formed by shockwaves and a powdered semiconductor material are further blended with the cosmetic product.

3. The cosmetic product recited in claim 2, wherein one or more of tourmaline, quartz and graphite is used as the infrared emitting material other than the diamond powder formed by shockwaves, and one or more of germanium and silicon is used as the semiconductor material.

4. The cosmetic product recited in claim 2, wherein a powdered composite material containing no less than 0.1 wt % and no more than 10 wt % of diamond powder is used.
